(19) United States
(12) Patent Application Publication
(10) Pub. No.: US 2018/0264058 A1
(43) Pub. Date: Sep. 20, 2018

(54) TRADITIONAL CHINESE MEDICINE COMPOSITION, PREPARATION METHOD AND USE THEREOF

(71) Applicant: INFINITUS (CHINA) COMPANY LTD., Jiang Men (CN)

(72) Inventors: Renhuai CONG, Jiang Men (CN); Fangli MA, Jiang Men (CN); Chung Wah MA, Jiang Men (CN); Xiaoling WANG, Jiang Men (CN); Lingyun XIAO, Jiang Men (CN)

(73) Assignee: INFINITUS (CHINA) COMPANY LTD., Jiang Men (CN)

(21) Appl. No.: 15/792,530

(22) Filed: Oct. 24, 2017

(30) Foreign Application Priority Data
Mar. 15, 2017 (CN) 201710153757.8

(51) Int. Cl. A61K 36/076; A61K 36/232; A61K 36/752; A61K 36/284

(52) U.S. Cl. CPC A61K 36/076; A61K 36/284; A61K 36/752; A61K 36/232

(57) ABSTRACT

The present disclosure relates to the field of health food technology, in particular to a traditional Chinese medicine composition, preparation method and use thereof. The traditional Chinese medicine composition is made from Poria, Angelicae Sinensis Radix, Aurantii Fructus Immaturus and Atractylodis Macrocephalae Rhizoma. The traditional Chinese medicine composition uses less species of Chinese herbs and has significant effect on reducing the circumference of waist and abdomen, reducing the weight of visceral fat and anti-inflammation by reasonable combination, and does not have side effects.

### CROSS-REFERENCE TO RELATED APPLICATIONS

The present application claims a priority to Chinese patent application No. 201710153757.8, as filed on Mar. 15, 2017 and titled with “TRADITIONAL CHINESE MEDICINE COMPOSITION, PREPARATION METHOD AND USE THEREOF”, and the disclosure of which is incorporated herein by reference.

### FIELD

The present disclosure relates to the field of health food technology, in particular to a traditional Chinese medicine composition, preparation method and use thereof.

### BACKGROUND

Abdominal obesity mainly refers to fat deposition in the abdominal cavity, manifesting an increase in the circumference of waist and abdomen. There are many factors that affect the increase in the circumference of waist and abdomen, such as unhealthy eating habits, lack of physical activity, being sedentary due to occupational habits, etc. The risk of developing complications in people with abdominal obesity is much greater than that in people with systemic obesity. Moreover, intra-abdominal fat is very different from subcutaneous fat. It is more harmful than subcutaneous fat, and it releases more harmful factors than subcutaneous fat does. Abdominal obesity is likely to damage internal organs, makes people more likely to suffer from high blood pressure, diabetes, hyperlipidemia, cholelithiasis, hyperuricemia and other diseases, so the detriment is severer. In addition, it was reported in American Journal of Clinical Research that a research group (Sano Yuanakijun, et al.) of Keio University have found that the active immune response in the visceral adipose tissue causes excess inflammatory response, of which the influence spread to the whole body. Abdominal obesity is the main type of obesity among Chinese people. A follow-up observation of 2778 subjects by the Medical College of Soochow University also have found that at the same time that BMIs of some individual decreases, their waist circumferences may not significantly decrease, or even may increase; while at the same time that the BMIs rise, the waist circumstances may reduce. Therefore, determining whether a person is obese by his or her weigh only is a misconception. It is likely to classify people with normal BMI but high waist into low-risk groups, but in fact, disease risk in this part of the people is still high. Therefore, controlling the abdominal obesity is of great significance.

So far, people mainly use exercise and drugs to lose weight. Mostly, once the exercise stops, it is likely to gain the weight back. Losing weight by drugs is mainly through suppressing central appetite, developing anorexia and satiety, reducing nutrient absorption, increasing energy consumption, etc., it usually has certain side effects, such as diarrhea, arrhythmia, and even liver damage and kidney damage, etc. Therefore, people look forward to the development of traditional Chinese medicine products having simple components, low toxicity and minor side effects. However, for the treatment of abdominal obesity, traditional Chinese medicine products have been rarely reported.

Patent Publication No. CN103070924A discloses a traditional Chinese medicine composition for treating metabolic syndrome, preparation method and use thereof. Said traditional Chinese medicine composition is prepared by using the following traditional Chinese medicinal materials as raw materials: by weight, Ilicis Cornutae Folium 6-30 parts, Nelumbinis Folium 6-30 parts, Moli Folium 6-30 parts. The traditional Chinese medicine composition or said traditional Chinese medicine composition with pharmaceutically acceptable carrier may be used to prepare pharmaceutical preparations for the treatment of metabolic syndrome, in particular to prepare pharmaceutical preparations for the treatment of abdominal obesity, type-II diabetes, hyperlipidemia or atherosclerotic disease. There is still a need for a new type of traditional Chinese medicine composition to treat abdominal obesity effectively.

### SUMMARY

In view of the above, the present disclosure provides a traditional Chinese medicine composition, preparation method and use thereof. The traditional Chinese medicine composition can reduce the circumference of waist and abdomen, reduce the weight of visceral fat, effectively improve the concentrations of inflammation-related factors IL-10, IL-18, PAI-1, adiponectin, TNF-α, IL6, CRP, etc. in serum. It can be used to prevent or treat abdominal obesity, and its effect is significantly better than the effect of each single herb.

In order to achieve the object of the present disclosure, the following technical solution(s) is provided in the present disclosure.

The present disclosure provides a traditional Chinese medicine composition, which is made from Poria, Angelicae Sinensis Radix, Aurantii Fructus Immaturus and Atractyodis Macrocephalae Rhizoma.

Traditional Chinese medicine theory in “Plain Questions⋅Qibinglun” said that “eating too many sweet and delicious foods would cause obesity.” Eating too many sweet and delicious foods, gradually accumulate and be converted to fat. Or uncontrolled diet damages spleen and stomach as time goes by, the essence of water and food cannot be transported or transformed normally, water-dampness detents and accumulates, dampness is engendered from the internal, dampness accumulates, phlegm is engendered, causes the reduction of muscle and the increase of fat which stop in skin as well as viscera and bowels, effuses and develops obesity. If overeat greasy and delicious foods, and lack exercise, or even sit or lie for long periods of time, causes the essence of nutrition in the body cannot be consumed, definitely accumulation will occur and fats will develop in the course of time.

The nature of Poria is sweet, bland and neutral. Its meridian tropism is in heart, spleen and kidney. Functions: induce diuresis to alleviate edema, drain dampness, fortify the spleen and quiets the heart.

The nature of Angelicae Sinensis Radix is sweet, acrid and warm. Its meridian tropism is in liver, heart and spleen. Functions: activate blood to regulate menstruation, active blood to relieve pain, moisten the intestines and relax the bowels.

The nature of Aurantii Fructus Immaturus is bitter, acrid and sour. Its meridian tropism is in spleen, stomach and large intestine. Functions: break Qi, relieve stuffiness, purge phlegm, and eliminate accumulation. It treats chest and abdominal distension and fullness, food accumulation, constipation and gastroptosis.

The nature of Atractylodis Macrocephalae Rhizoma is sweet, bitter and warm. Its meridian tropism is in spleen and stomach. Functions: fortify the spleen, tonify Qi, dry dampness, increases urine excretion, arrests excessive sweating and prevent abortion.

The combination of Chinese herbs mentioned above can drain dampness, resolve phlegm, transport spleen Qi, at the same time it can activate blood, free the collateral vessels, soothe the liver and regulate Qi, and free Qi movement. It was proved by the tests in the present disclosure that they can reduce the circumference of waist and abdomen, reduce the weight of visceral fat, and effectively improve the concentrations of inflammation-related factors IL-10, IL-18, PAI-1, adiponectin, TNF-α, IL6, CRP, etc. in serum. It may be used to prevent or treat abdominal obesity, and its effect is significantly better than the effect of each single herb in the combination.

Preferably, by weight, the dosage of each raw material is:

Poria 10-90 parts;

Angelicae Sinensis Radix 5-60 parts;

Aurantii Fructus Immaturus 5-60 parts;

Atractylodis Macrocephalae Rhizoma 5-60 parts.

Preferably, by weight, the dosage of each raw material is:

Poria 20-90 parts;

Angelicae Sinensis Radix 10-40 parts;

Aurantii Fructus Immaturus 10-40 parts;

Atractylodis Macrocephalae Rhizoma 10-40 parts.

More preferably, by weight, the dosage of each raw material is:

Poria 30 parts;

Angelicae Sinensis Radix 20 parts;

Aurantii Fructus Immaturus 20 parts;

Atractylodis Macrocephalae Rhizoma 20 parts.

In one example of the present disclosure, by weight, the dosage of each raw material is:

Poria 60 parts;

Angelicae Sinensis Radix 10 parts;

Aurantii Fructus Immaturus 40 parts;

Atractylodis Macrocephalae Rhizoma 10 parts.

In another example of the present disclosure, by weight, the dosage of each raw material is:

Poria 20 parts;

Angelicae Sinensis Radix 40 parts;

Aurantii Fructus Immaturus 10 parts;

Atractylodis Macrocephalae Rhizoma 40 parts.

In another example of the present disclosure, by weight, the dosage of each raw material is:

Poria 90 parts;

Angelicae Sinensis Radix 10 parts;

Aurantii Fructus Immaturus 10 parts;

Atractylodis Macrocephalae Rhizoma 10 parts.

The present disclosure also provides uses of the traditional Chinese medicine composition(s) in the preparation of medicaments and health foods for treating abdominal obesity.

In the embodiments provided by the present disclosure, treating abdominal obesity refers to one or more of reducing the circumference of waist and abdomen, decreasing Lee's index, reducing the weight of visceral fat, or improving the concentrations of inflammation-related factor(s) IL-10, IL-18, PAI-1, adiponectin, TNF-α, IL6 and CRP in serum.

The present disclosure also provides a method for preparing the traditional Chinese medicine composition, comprising: mixing Poria, Angelicae Sinensis Radix, Aurantii Fructus Immaturus and Atractylodis Macrocephalae Rhizoma, adding water, soaking, decocting, filtering, and obtaining the traditional Chinese medicine composition.

Preferably, the quantity of water is 3-10 times as much as of the total weight of raw materials; soaking time is 0.25 h-2 h; decoction number is 1-5 times; time of each decoction is 1 h-4 h.

In the embodiments of the present disclosure, the quantity of water is 8 times as much as the total weight of raw materials; soaking time is 1 h; decoction number is 2 times; time of each decoction is 2 h.

Preferably, a concentration step(s) is also included after the filtration.

In the embodiments of the present disclosure, the concentration is: concentrating until soluble solids concentration is 80%.

The present disclosure also provides a type of pharmaceutical, comprising the traditional Chinese medicine composition provided by the present disclosure.

Preferably, the pharmaceutical(s) also comprises pharmaceutically acceptable excipient(s).

Preferably, the dosage forms of the pharmaceutical(s) are paste, granules, pill, powder, tablet, capsule, oral agent or syrup. However, the dosage form of the pharmaceutical(s) is not limited thereto, any dosage form considered by those of ordinary skill in the art as a feasible one is within the scope of the present disclosure.

The present disclosure also provides a health food, comprising the traditional Chinese medicine composition(s) provided in the present disclosure.

Preferably, the health foods further comprise food-acceptable food additive(s).

Preferably, the dosage forms of the health foods are granule, capsule, syrup, tablet, powder, gummy candy, emulsion or oral solution. However, the dosage form of the health foods is not limited thereto, any dosage form considered by those of ordinary skill in the art as a feasible one is within the scope of the present disclosure.

The present disclosure provides a traditional Chinese medicine composition, preparation method and use thereof. The traditional Chinese medicine composition is made from Poria, Angelicae Sinensis Radix, Aurantii Fructus Immaturus and Atractylodis Macrocephalae Rhizoma. The technical effects of the present disclosure are:

The traditional Chinese medicine composition of the present disclosure is made from raw materials comprising Poria, Angelicae Sinensis Radix, Aurantii Fructus Immaturus and Atractylodis Macrocephalae Rhizoma. The traditional Chinese medicine composition uses less specie(s) of Chinese herbs and has significant effects on reducing the circumference of waist and abdomen, reducing the weight of visceral fat, and anti-inflammation by reasonable combination, and does not have side effects. The results of tests showed that the traditional Chinese medicine composition(s) of the present disclosure can reduce the circumference of waist and abdomen, reduce the weight of visceral fat, and effectively improve the concentrations of inflammation-related factors IL-10, IL-18, PAI-1, adiponectin, TNF-α, IL6, CRP, in serum. It may be used to prevent or treat abdominal obesity, and its effect is significantly better than the effect of each single herb.

### DETAILED DESCRIPTION OF EMBODIMENTS

The present disclosure discloses a traditional Chinese medicine composition, preparation method and use thereof, and those of ordinary skill in the art may achieve by properly improving process parameter(s) using the contents of this disclosure. In particular, it needs to be pointed out that all similar substitutions and modifications are apparent to those of ordinary skill in the art and are considered to be included in the present disclosure. The method(s) and use(s) of the present disclosure have been described by way of preferred embodiments, it is apparent that those of ordinary skill in the art are able to change or properly modify and combine said method(s) and use(s) herein without departing from the content, spirit and scope of the present disclosure to achieve and use the techniques of the present disclosure.

The raw materials and excipients used in the traditional Chinese medicine composition, preparation method and use thereof provided by the present disclosure are all commercially available.

The present disclosure is further described in conjunction with the following Examples:

### Example 1

### Preparation of the Traditional Chinese Medicine Composition

### 1. Raw Materials

The weight proportion of every raw materials: Poria 30 parts, Angelicae Sinensis Radix 20 parts, Aurantii Fructus Immaturus 20 parts, Atractylodis Macrocephalae Rhizoma 20 parts.

### 2. Preparation Method

The active pharmaceutical ingredients of Poria, Angelicae Sinensis Radix, Aurantii Fructus Immaturus and Atractylodis Macrocephalae Rhizoma were mixed, soaked in added water, and the amount of water added was 8 times as much as the weight of the active pharmaceutical ingredients of raw materials, and the soaking time was 1 h. Decoction was performed twice, 2 h each time. The decoction solution was filtered and concentrated until solids content was 80%, said traditional Chinese medicine composition of the present disclosure was obtained.

### Example 2

### Preparation of the Traditional Chinese Medicine Composition

### 1. Raw Materials

The weight proportion of every raw materials: Poria 60 parts, Angelicae Sinensis Radix 10 parts, Aurantii Fructus Immaturus 40 parts, Atractylodis Macrocephalae Rhizoma 10 parts.

### 2. Preparation Method

The preparation method was the same as that in Example 1.

### Example 3

### Preparation of the Traditional Chinese Medicine Composition

### 1. Raw Materials

The weight proportion of every raw materials: Poria 20 parts, Angelicae Sinensis Radix 40 parts, Aurantii Fructus Immaturus 10 parts, Atractylodis Macrocephalae Rhizoma 40 parts.

### 2. Preparation Method

The preparation method was the same as that in Example 1.

### Example 4

### Preparation of the Traditional Chinese Medicine Composition

### 1. Raw Materials

The weight proportion of every raw materials: Poria 90 parts, Angelicae Sinensis Radix 10 parts, Aurantii Fructus Immaturus 10 parts, Atractylodis Macrocephalae Rhizoma 10 parts.

### 2. Preparation Method

The preparation method was the same as that in Example 1.

### Example 5

### Pharmacodynamic Experiment of the Traditional Chinese Medicine Compositions for

### Treating Abdominal Obesity

### 1. Experimental Materials

### (1) Main Reagents

Feed (whole milk protein, corn starch, sucrose, soybean oil), minerals, vitamins, raw materials were self-purchased, were completed by the preparation method of Hubei University of Traditional Chinese Medicine, were completed by the processing of the Experimental Animal Center of Hubei University of Traditional Chinese Medicine.

Chlorpromazine injection, Southwest Pharmaceutical Co., Ltd., national medicine permission number H50020116.

Kit, Nanjing Jiancheng Bioengineering Institute.

### (2) Animals

SD rats, male, SPF grade, body weight (180±15) g, provided by the Animal Experimental Center of Hubei University of Traditional Chinese Medicine.

### (3) Main Instruments

Electronic analytical balance, BS124S, Startorius (Germany); microplate reader, Bio-Rad Laboratories, Inc. (USA); ultralow-temperature freezer, Thermo Fisher Scientific Inc. (USA); ultra pure water machine, Mill-Q II, Milipore (Bedford, Mass., USA); DDL-5 freezing centrifuge, Shanghai Anting Scientific Instrument Factory.

### 2. Test Methods

Healthy male SD rats were housed in normal maintenance feed (equivalent to normal diet) for 3 days.

Blank group, model group, test group were set up respectively. The blank control group was given intraperitoneal injection of saline once and freely drank and ate; the model group and each test group were all injected intraperitoneally with chlorpromazine 3 mg/kg once and freely drank and ate. During the experiments, the blank group was given the basic feed; the model group and the test groups were given medium-fat feed (including: 140 g/kg whole milk protein, corn starch 538.1 g/kg, sucrose 87.6 g/kg, soybean oil 137 g/kg), and minerals and vitamins (minerals 35 g/kg, vitamin 50 g/kg, choline 2.3 g/kg) were complemented into it. All of them continued 15 days.

After the model was set up, each test group took the test sample for 6 weeks: the low, medium and high dosage groups of Example 1 (4.5:9.0:18.0 g/kg), high dosage group (18.0 g/kg) of Examples 2, 3 and 4, the administration volume was 2.0 mL/kg. The model group was given the corresponding dosage of distilled water.

### 3. Test Indexes

After 6 weeks, the animals were sacrificed, blood was taken and dissection was performed. The circumference of waist and abdomen, body weight and body length of the rats were measured accurately and the Lee's index was calculated.

Perinephric fat, epididymal fat, epicardial fat, retroperitoneal fat, greater omental fat and mesenteric fat, and total visceral adipose tissue (VAT) were weighed.

The concentrations of inflammation-related factors in serum: PAI-1 (Plasminogen Activator Inhibitor), adiponectin, CRP, TNF-α, IL-6, serum testosterone were determined.

### 4. Statistical Methods

In general, the analysis of variance was used; however, it was needed that the homogeneity of variance test was firstly performed according to the variance analysis procedure. If the variance was homogeneous, the F value was calculated. F<F_{0.05}, conclusion: the difference between the averages of each group was not significant. F≥F_{0.05}, p≤0.05, statistical analysis was performed by the pairwise comparison between the average of multiple experimental groups and the average of control group. For non-normal or non-homogeneous data, appropriate transformation(s) of variables was carried out. The transformed data was subjected to statistical analysis after meeting the normal or homogeneous variance requirements. If the transformed data still did not reach the normal or homogeneous variance after the transformation of variables, statistical analysis was performed by rank sum test.

### 5. Results of the Experiment

After the completion of the experiments, the indicator level changes of each group were shown in Table 1 to Table 3.

TABLE 1
The effects of each dosage group on Lee's index and
circumference of waist and abdomen of rats

| | | | Circumference of |
|---|---|---|---|
| | Number | | waist and |
| Group | of cases | Lee's index | abdomen (cm) |
| Blank group | 15 | 293.61 ± 1.08 | 14.25 ± 0.05 |
| Model group | 15 | 309.18 ± 1.22^{#} | 17.90 ± 0.10^{#} |
| Low dosage group of | 15 | 300.76 ± 6.23^{※} | 16.15 ± 0.05^{※} |
| Example 1 | | | |
| Medium dosage group of | 15 | 294.84 ± 8.61^{※} | 15.75 ± 0.35^{※} |
| Example 1 | | | |
| High dosage group of | 15 | 294.26 ± 9.33^{※} | 15.25 ± 0.15^{※} |
| Example 1 | | | |
| High dosage group of | 15 | 294.67 ± 0.22^{※} | 15.11 ± 0.54^{※} |
| Example 2 | | | |
| High dosage group of | 15 | 295.33 ± 1.01^{※} | 15.36 ± 0.36^{※} |
| Example 3 | | | |
| High dosage group of | 15 | 296.04 ± 1.67^{※} | 15.27 ± 0.27^{※} |
| Example 4 | | | |

Note:
^{#}compared with the blank group, p < 0.05;
^{※}compared with the model group, p < 0.05.

The comparisons of Lee's index of each rat group are shown in Table 1. Comparing the Lee's index of the rats in the model group with that in the blank group, the difference was statistically meaningful (p<0.05). There was statistical difference between the Lee's index in each sample group and the model group (p<0.05). There was no statistical difference among sample groups (p>0.05). As to the index of circumference of waist and abdomen, there was statistical difference comparing the sample groups with the model group respectively (p<0.05).

TABLE 2
The comparisons of body fat (g) of each rat group

| | Retroperitoneal | Epididymal | Epicardial | Greater omental | Perinephric | Mesenteric | Visceral adipose |
|---|---|---|---|---|---|---|---|
| Group | fat | fat | fat | fat | fat | fat | tissue (VAT) |
| Blank group | 2.33 ± 0.60 | 1.58 ± 0.29 | 0.31 ± 0.09 | 0.98 ± 0.09 | 0.35 ± 0.05 | 2.56 ± 0.20 | 8.11 ± 0.32 |
| Model group | 3.98 ± 0.65^{#} | 1.95 ± 0.38^{#} | 0.39 ± 0.23^{#} | 1.39 ± 0.31^{#} | 0.42 ± 0.14 | 4.10 ± 1.10^{#} | 13.39 ± 2.61^{#} |
| Low dosage group | 3.20 ± 1.22^{※} | 1.66 ± 0.95^{※} | 0.37 ± 0.43 | 1.37 ± 0.14 | 0.36 ± 0.02 | 3.13 ± 0.24 | 10.11 ± 2.08^{※} |
| of Example 1 | | | | | | | |
| Medium dosage group | 2.93 ± 1.16^{※} | 1.640 ± 0.02^{※} | 0.31 ± 0.12 | 1.29 ± 0.05^{※} | 0.39 ± 0.25 | 2.26 ± 0.99^{※} | 9.21 ± 2.05^{※} |
| of Example 1 | | | | | | | |
| High dosage group | 2.77 ± 0.82^{※} | 1.56 ± 0.98^{※} | 0.29 ± 0.10^{※} | 1.28 ± 0.15^{※} | 0.35 ± 0.28^{※} | 2.54 ± 0.18^{※} | 8.90 ± 1.73^{※} |
| of Example 1 | | | | | | | |
| High dosage group | 2.89 ± 0.23^{※} | 1.57 ± 0.21^{※} | 0.30 ± 0.03^{※} | 1.29 ± 0.26^{※} | 0.35 ± 0.23^{※} | 2.72 ± 0.53^{※} | 9.15 ± 1.24^{※} |
| of Example 2 | | | | | | | |
| High dosage group | 2.79 ± 0.25^{※} | 1.56 ± 0.42^{※} | 0.30 ± 0.17^{※} | 1.29 ± 0.19^{※} | 0.37 ± 0.09^{※} | 2.68 ± 0.48^{※} | 9.31 ± 2.34^{※} |
| of Example 3 | | | | | | | |
| High dosage group | 2.91 ± 0.33^{※} | 1.56 ± 0.25^{※} | 0.31 ± 0.09^{※} | 1.30 ± 0.14^{※} | 0.36 ± 0.23^{※} | 2.76 ± 0.50^{※} | 9.55 ± 1.27^{※} |
| of Example 4 | | | | | | | |

Note:
^{#}compared with the blank group, p < 0.05;
^{※}compared with the model group, p < 0.05.

There were statistical differences comparing the weight of retroperitoneal fat, epididymal fat, epicardial fat, greater omental fat, mesenteric fat and total visceral adipose tissue (VAT) of the rats in the model group with those in the blank group (p<0.05), which indicated the success of modeling.

Comparing each sample group with the model group, there were statistical meaning in part of indexes of weight of retroperitoneal fat, epicardial fat, greater omental fat, perinephric fat, epididymal fat and mesenteric fat (p<0.05).

As to the indexes of weight of retroperitoneal fat, epididymal fat, greater omental fat, mesenteric fat and total visceral adipose tissue (VAT), there were statistical differences comparing the medium and high dosage group of Example 1 with the model group (p<0.05).

As to the indexes of weight of retroperitoneal fat, epicardial fat, greater omental fat, perinephric fat, epididymal fat and mesenteric fat as well as visceral adipose tissue (VAT), there was statistical differences comparing the high dosage group of Example 1 and the high dosage groups of Examples 2 to 4 of the sample group with the model group (p<0.05).

TABLE 3
The comparisons of inflammation-related factors of each group

| | PAI-1 | Adiponectin | CRP | TNF-α | IL-6 | Serum testosterone |
|---|---|---|---|---|---|---|
| Group | (ng/ml) | (ng/ml) | (ng/ml) | (pg/ml) | (pg/ml) | (ng/ml) |
| Blank group | 16.83 ± 0.26 | 19.535 ± 10.524 | 1.256 ± 0.262 | 200.117 ± 25.062 | 96.068 ± 9.869 | 9.09 ± 1.99 |
| Model group | 40.67 ± 5.22^{#} | 4.554 ± 0.276^{#} | 5.381 ± 0.253^{#} | 530.200 ± 68.038^{#} | 239.757 ± 17.210^{#} | 1.10 ± 0.12^{#} |
| Low dosage group | 31.16 ± 5.16 | 5.916 ± 0.928 | 4.157 ± 0.176^{※} | 379.869 ± 3.962^{※} | 203.477 ± 10.710^{※} | 1.47 ± 0.29 |
| of Example 1 | | | | | | |
| Medium dosage group | 27.43 ± 0.92^{※} | 8.861 ± 0.316^{※} | 3.567 ± 0.818^{※} | 342.438 ± 28.841^{※} | 184.499 ± 14.978^{※} | 3.12 ± 0.69^{※} |
| of Example 1 | | | | | | |
| High dosage group | 26.89 ± 0.89^{※} | 11.052 ± 1.433^{※} | 3.494 ± 0.894^{※} | 290.481 ± 34.361^{※} | 181.445 ± 24.766^{※} | 3.94 ± 0.51^{※} |
| of Example 1 | | | | | | |
| High dosage group | 25.25 ± 1.02^{※} | 11.234 ± 1.112^{※} | 3.638 ± 0.765^{※} | 280.332 ± 20.098^{※} | 190.876 ± 13.244^{※} | 4.01 ± 0.26^{※} |
| of Example 2 | | | | | | |
| High dosage group | 26.59 ± 1.00^{※} | 10.923 ± 0.347^{※} | 3.567 ± 0.528^{※} | 287.481 ± 19.754^{※} | 188.322 ± 24.766^{※} | 3.96 ± 0.44^{※} |
| of Example 3 | | | | | | |
| High dosage group | 26.41 ± 0.90^{※} | 9.934 ± 0.593^{※} | 3.433 ± 0.234^{※} | 300.764 ± 30.001^{※} | 192.445 ± 10.766^{※} | 3.61 ± 0.37^{※} |
| of Example 4 | 26.23 ± 0.88^{※} | | | | | |

Note:
^{#}compared with the blank group, p < 0.05;
^{※}compared with the model group, p < 0.05.

Comparing the concentrations of PAI-1, adiponectin, CRP, TNF-α, IL-6 and serum testosterone of the rats in the model group with those in the blank group, all the differences were statistically meaningful, (P<0.05), which indicated the success of modeling.

Comparing the concentrations of PAI-1, adiponectin, CRP, TNF-α, IL-6 and serum testosterone of each sample group with those in the model group, there were significant differences (p<0.05).

### 6. Summary of the Experiment

Comprehensive analysis of the above data shows that all of the sample groups can reduce the circumference of waist and abdomen, and eliminate the visceral fat to varying degrees. There are significant differences comparing the sample groups with the model group.

Compared with the blank group, inflammation-related factors PAI-1, adiponectin, CRP, TNF-α, IL-6, serum testosterone, etc. in serum of the model group all show significant increases or decreases, while each sample group can improve the change(s) of these factors to certain degrees. It indicates that each sample group has an intervention effect on the imbalance of inflammation-related factors in abdominal obesity model.

### Example 6

### Pharmacodynamic Experiment of the Traditional Chinese Medicine Compositions for Treating Abdominal Obesity

### 1. Experimental Materials

### (1) Main Reagents

Feed (whole milk protein, corn starch, sucrose, soybean oil), minerals, vitamins, raw materials were self-purchased, were completed by the preparation method of Hubei University of Traditional Chinese Medicine, were completed by the processing of the Experimental Animal Center of Hubei University of Traditional Chinese Medicine.

Chlorpromazine injection, Southwest Pharmaceutical Co., Ltd., national medicine permission number H50020116.

Kit, Nanjing Jiancheng Bioengineering Institute.

### (2) Animals

SD rats, male, SPF grade, body weight (180±15) g, provided by the Animal Experimental Center of Hubei University of Chinese Medicine.

### (3) Main Instruments

Electronic analytical balance, BS124S, Startorius (Germany); microplate reader, Bio-Rad Laboratories, Inc. (USA); ultralow-temperature freezer, Thermo Fisher Scientific Inc. (USA); ultra pure water machine, Mill-Q II, Milipore (Bedford, Mass., USA); DDL-5 freezing centrifuge, Shanghai Anting Scientific Instrument Factory.

### 2. Test Methods

Healthy male SD rats were housed in normal maintenance feed (equivalent to normal diet) for 3 days.

Blank group, model group, test group were set up respectively. The blank control group was given intraperitoneal injection of saline once and freely drank and ate; the model group and each test group were injected intraperitoneally with chlorpromazine 3 mg/kg once and freely drank and ate. During the experiment, the blank group was given basic feed; the model group and the test groups were given medium-fat feed (including: 140 g/kg whole milk protein, corn starch 538.1 g/kg, sucrose 87.6 g/kg, soybean oil 137 g/kg), and minerals and vitamins (minerals 35 g/kg, vitamin 50 g/kg, choline 2.3 g/kg) were complemented into it. All of them continued 15 days.

After the model was set up, each test group took the test sample for 6 weeks: the high dosage group of Example 1 (18.0 g/kg), single herb group of Poria, Angelicae Sinensis Radix, Aurantii Fructus Immaturus and Atractylodis Macrocephalae Rhizoma (18.0 g/kg, preparation method of the single herb was the same as that in Example 1). The administration volume was 2.0 mL/kg. The model group was given corresponding dosage of distilled water.

### 3. Test Indexes

After 6 weeks, the animals were sacrificed, blood was taken and dissection was performed. The circumference of waist and abdomen, body weight and body length of rats were measured accurately and Lee's index was calculated.

Perinephric fat, epididymal fat, epicardial fat, retroperitoneal fat, greater omental fat, mesenteric, and total visceral adipose tissue (VAT) were weighed.

The concentrations of inflammation-related factors in serum: PAI-1 (Plasminogen Activator Inhibitor), adiponectin, CRP, TNF-α, IL-6, and serum testosterone were determined.

### 4. Statistical Methods

In general, the analysis of variance was used; however, it was need that the homogeneity of variance test was firstly performed according to the variance analysis procedure. If the variance was homogeneous, the F value was calculated. F<F_{0.05}, conclusion: the difference between the averages of each group was not significant. F≥F_{0.05}, p≤0.05, statistical analysis was performed by the pairwise comparison between the average of several experimental groups and the average of control group. For non-normal or non-homogeneous data, appropriate transformation(s) of variables was carried out. The transformed data was subjected to statistical analysis after meeting the normal or homogeneous variance requirements. If the transformed data still did not reach the normal or homogeneous variance after the transformation of variables, statistical analysis was performed by rank sum test.

### 5. Results of the Experiment

After the completion of the experiments, the index level changes of each group are shown in Table 4 to Table 6.

TABLE 4
The effects of each dosage group on Lee's index and
circumference of waist and abdomen of rats

| | | | Circumference |
|---|---|---|---|
| | Number of | | of waist and |
| Group | cases | Lee's index | abdomen (cm) |
| Blank group | 15 | 293.61 ± 1.08 | 14.25 ± 0.05 |
| Model group | 15 | 309.18 ± 1.22^{#} | 17.90 ± 0.10^{#} |
| Poria group | 15 | 303.22 ± 2.01^{※}* | 17.32 ± 0.11^{※}* |
| Angelicae Sinensis | 15 | 299.98 ± 1.65^{※}* | 16.91 ± 0.12^{※}* |
| Radix group | | | |
| Aurantii Fructus | 15 | 297.76 ± 1.67^{※}* | 15.95 ± 0.16^{※}* |
| Immaturus group | | | |
| Atractylodis | 15 | 302.18 ± 0.78^{※}* | 17.16 ± 0.09^{※}* |
| Macrocephalae | | | |
| Rhizoma group | | | |
| High dosage group of | 15 | 294.26 ± 9.33^{※}* | 15.25 ± 0.15^{※}* |
| Example 1 | | | |

Note:
^{#}compared with the blank group, p < 0.05;
^{※}compared with the model group, p < 0.05;
*compared with the high dosage group of Example 1, p < 0.05.

The comparisons of Lee's index of each rat group are shown in Table 4. Comparing each single herb group with the model group, the Lee's indexes were statistically different (p<0.05). As to the indexes of circumference of waist and abdomen, there were statistical differences between each single herb group and model group (p<0.05). Also, comparing the high dosage group of Example 1 with each single herb group, the Lee's indexes were statistically different (p<0.05). As to the indexes of circumference of waist and abdomen, there were statistical differences comparing the high dosage group of Example 1 with each single herb group.

TABLE 5
The comparisons of body fat (g) of each rat group

| | Retroperitoneal | Epididymal | Epicardial | Greater omental | Perinephric | Mesenteric | Visceral adipose |
|---|---|---|---|---|---|---|---|
| Group | fat | fat | fat | fat | fat | fat | tissue (VAT) |
| Blank group | 2.33 ± 0.60 | 1.58 ± 0.29 | 0.31 ± 0.09 | 0.98 ± 0.09 | 0.35 ± 0.05 | 2.56 ± 0.20 | 8.11 ± 0.32 |
| Model group | 3.98 ± 0.65^{#} | 1.95 ± 0.38^{#} | 0.39 ± 0.23^{#} | 1.39 ± 0.31^{#} | 0.42 ± 0.14 | 4.10 ± 1.10^{#} | 13.39 ± 2.61^{#} |
| Poria group | 3.67 ± 0.34* | 1.67 ± 0.29* | 0.33 ± 0.07^{※}* | 1.37 ± 0.19* | 0.37 ± 0.11 | 3.38 ± 0.37* | 13.33 ± 0.33* |
| Angelicae Sinensis | 3.45 ± 0.55* | 1.73 ± 0.40* | 0.34 ± 0.04* | 1.36 ± 0.03* | 0.39 ± 0.09 | 3.55 ± 0.38* | 12.11 ± 0.70* |
| Radix group | | | | | | | |
| Aurantii Fructus | 3.22 ± 0.52^{※}* | 1.62 ± 0.22^{※}* | 0.32 ± 0.06^{※}* | 1.34 ± 0.06^{※}* | 0.37 ± 0.15 | 3.67 ± 0.45* | 10.09 ± 1.21^{※}* |
| Immaturus group | | | | | | | |
| Atractylodis | 3.36 ± 0.12^{※}* | 1.69 ± 0.31* | 0.36 ± 0.01* | 1.35 ± 0.11^{※}* | 0.38 ± 0.02 | 3.45 ± 0.09* | 11.01 ± 0.61* |
| Macrocephalae | | | | | | | |
| Rhizoma group | | | | | | | |
| High dosage group | 2.77 ± 0.82^{※} | 1.56 ± 0.98^{※} | 0.29 ± 0.10^{※} | 1.28 ± 0.15^{※} | 0.35 ± 0.28^{※} | 2.54 ± 0.18^{※} | 9.39 ± 1.73^{※} |
| of Example 1 | | | | | | | |

Note:
^{#}compared with the blank group, p < 0.05;
^{※}compared with the model group, p < 0.05,
*compared with the high dosage group of Example 1, p < 0.05.

As to the indexes of weights of retroperitoneal fat, epididymal fat, epicardial fat, greater omental fat, mesenteric fat and total visceral adipose tissue (VAT), there were significant differences comparing the high dosage group of Example 1 with each single herb group (p<0.05).

TABLE 6
The comparisons of inflammation-related factors of each group

| | PAI-1 | Adiponectin | CRP | TNF-α | IL-6 | Serum testosterone |
|---|---|---|---|---|---|---|
| Group | (ng/ml) | (ng/ml) | (ng/ml) | (pg/ml) | (pg/ml) | (ng/ml) |
| Blank group | 16.83 ± 0.26 | 19.535 ± 10.524 | 1.256 ± 0.262 | 200.117 ± 25.062 | 96.068 ± 9.869 | 9.09 ± 1.99 |
| Model group | 40.67 ± 5.22^{#} | 4.554 ± 0.276^{#} | 5.381 ± 0.253^{#} | 530.200 ± 68.038^{#} | 239.757 ± 17.210^{#} | 1.10 ± 0.12^{#} |
| Poria group | 34.55 ± 3.21* | 5.889 ± 0.276* | 4.381 ± 0.208^{※}* | 400.232 ± 34.038^{※}* | 203.986 ± 11.210^{※}* | 1.47 ± 0.11* |
| Angelicas Sinensis | 36.67 ± 0.67* | 5.927 ± 0.276* | 4.110 ± 0.123^{※}* | 498.109 ± 33.038* | 210.118 ± 12.479^{※}* | 1.98 ± 0.33* |
| Radix group | | | | | | |
| Aurantii Fructus | 33.32 ± 3.33* | 6.001 ± 0.276* | 3.991 ± 0.198^{※}* | 432.667 ± 17.098^{※}* | 210.723 ± 9.854^{※}* | 1.12 ± 0.19* |
| Immaturus group | | | | | | |
| Atractylodis | 36.87 ± 4.11* | 6.546 ± 0.276^{※}* | 4.210 ± 0.234^{※}* | 468.286 ± 18.987^{※}* | 219.092 ± 21.234* | 1.95 ± 0.08* |
| Macrocephalae | | | | | | |
| Rhizoma group | | | | | | |
| High dosage group | 26.89 ± 0.89^{※} | 11.052 ± 1.433^{※} | 3.494 ± 0.894^{※} | 290.481 ± 34.361^{※} | 181.445 ± 24.766^{※}* | 3.94 ± 0.51^{※} |
| of Example 1 | | | | | | |

Note:
^{#}compared with the blank group, p < 0.05;
^{※}compared with the model group, p < 0.05;
*compared with the high dosage group of Example 1, p < 0.05.

Comparing the concentrations of PAI-1, adiponectin, CRP, TNF-α, IL-6 and serum testosterone of the Example 1 group with those in each single herb group, all of the differences were statistically meaningful (p<0.05).

### 6. Summary of the Experiment

Comprehensive analysis of the above data shows that all example groups can reduce the circumference of waist and abdomen, and eliminate the visceral fat to varying degrees. There are significant differences comparing with each single herb group.

The inflammation-related factors PAI-1, adiponectin, CRP, TNF-α, IL-6, serum testosterone, etc. all show significant increases or decreases in serum in the example groups, the improvement function of sample group is better than that of each single herb group. These results indicate that the traditional Chinese medicine composition(s) of the present disclosure has an intervention effect on the imbalance of inflammation-related factors in abdominal obesity model.

### Comparative Example 1

The experimental method was referred to the methods in Example 6, pharmacodynamics of multiple combinations of each traditional Chinese medicine extracts were investigated. Results are shown in Table 7 to Table 9.

TABLE 7
The effects of each dosage group on Lee's index and circumference of waist and
abdomen of rats

| | | Number of | | Circumference of waist |
|---|---|---|---|---|
| No. | Group | cases | Lee's index | and abdomen(cm) |
| 1 | Blank group | 15 | 293.61 ± 1.08 | 14.25 ± 0.05 |
| 2 | Model group | 15 | 309.18 ± 1.22^{#} | 17.90 ± 0.10^{#} |
| 3 | High dosage group of Example 1 | 15 | 294.26 ± 9.33 | 15.25 ± 0.15 |
| 4 | Poria 30 parts + Angelicae Sinensis Radix 20 parts | 15 | 300.76 ± 6.23^{Δ} | 16.15 ± 0.05^{Δ} |
| 5 | Poria 30 parts + Aurantii Fructus Immaturus 20 parts | 15 | 300.10 ± 1.37^{Δ} | 16.07 ± 0.06^{Δ} |
| 6 | Poria 30 parts + Atractylodis Macrocephalae Rhizoma | 15 | 300.23 ± 1.93^{Δ} | 16.09 ± 0.63^{Δ} |
| | 20 parts | | | |
| 7 | Angelicae Sinensis Radix 20 parts + Aurantii Fructus | 15 | 300.98 ± 7.67^{Δ} | 16.15 ± 0.62^{Δ} |
| | Immaturus 20 parts | | | |
| 8 | Angelicae Sinensis Radix 20 parts + Atractylodis | 15 | 299.46 ± 3.67^{Δ} | 16.05 ± 0.16^{Δ} |
| | Macrocephalae Rhizoma 20 parts | | | |
| 9 | Aurantii Fructus Immaturus 20 parts + Atractylodis | 15 | 299.05 ± 2.67^{Δ} | 15.99 ± 0.90^{Δ} |
| | Macrocephalae Rhizoma 20 parts | | | |
| 10 | Poria 30 parts + Angelicae Sinensis Radix 20 parts + | 15 | 298.22 ± 4.23^{Δ} | 15.85 ± 0.09^{Δ} |
| | Aurantii Fructus Immaturus 20 parts | | | |
| 11 | Poria 30 parts + Angelicae Sinensis Radix 20 parts + | 15 | 297.99 ± 1.24^{Δ} | 15.89 ± 0.13^{Δ} |
| | Atractylodis Macrocephalae Rhizoma 20 parts | | | |
| 12 | Angelicae Sinensis Radix 20 parts + Aurantii Fructus | 15 | 297.23 ± 2.74^{Δ} | 15.96 ± 0.12^{Δ} |
| | Immaturus 20 parts + Atractylodis Macrocephalae | | | |
| | Rhizoma 20 parts | | | |
| 13 | Poria 30 parts + Aurantii Fructus Immaturus 20 parts + | 15 | 296.98 ± 3.38^{Δ} | 15.97 ± 0.10^{Δ} |
| | Atractylodis Macrocephalae Rhizoma 20 parts | | | |

Note:
^{Δ}compared with the high dosage group of Example 1, p < 0.05.

TABLE 8
The comparisons of body fat (g) of each rat group

| | Retroperitoneal | Epididymal | Epicardial | Greater omental | Perinephric | Mesenteric | Visceral adipose |
|---|---|---|---|---|---|---|---|
| No. | fat | fat | fat | fat | fat | fat | tissue (VAT) |
| 1 | 2.33 ± 0.60 | 1.58 ± 0.29 | 0.31 ± 0.09 | 0.98 ± 0.09 | 0.35 ± 0.05 | 2.56 ± 0.20 | 8.11 ± 0.32 |
| 2 | 3.98 ± 0.65^{#} | 1.95 ± 0.38^{#} | 0.39 ± 0.23^{#} | 1.39 ± 0.31^{#} | 0.42 ± 0.14 | 4.10 ± 1.10^{#} | 13.39 ± 2.61^{#} |
| 3 | 2.77 ± 0.82 | 1.56 ± 0.98 | 0.29 ± 0.10 | 1.28 ± 0.15 | 0.35 ± 0.28 | 2.54 ± 0.18 | 9.39 ± 1.73 |
| 4 | 3.22 ± 1.21^{Δ} | 1.66 ± 0.18^{Δ} | 0.37 ± 0.11^{Δ} | 1.37 ± 0.11^{Δ} | 0.36 ± 0.02 | 3.63 ± 0.24^{Δ} | 10.11 ± 1.08^{Δ} |
| 5 | 3.22 ± 0.19^{Δ} | 1.65 ± 0.34^{Δ} | 0.36 ± 0.01^{Δ} | 1.37 ± 0.05^{Δ} | 0.37 ± 0.15 | 3.67 ± 0.23^{Δ} | 10.09 ± 1.21^{Δ} |
| 6 | 3.22 ± 0.12^{Δ} | 1.66 ± 0.23^{Δ} | 0.37 ± 0.02^{Δ} | 1.36 ± 0.06^{Δ} | 0.36 ± 0.05 | 3.53 ± 0.17^{Δ} | 10.11 ± 0.08^{Δ} |
| 7 | 3.11 ± 0.07^{Δ} | 1.64 ± 0.23^{Δ} | 0.35 ± 0.01^{Δ} | 1.36 ± 0.12^{Δ} | 0.35 ± 0.02 | 3.53 ± 0.16^{Δ} | 10.10 ± 0.52^{Δ} |
| 8 | 3.21 ± 0.09^{Δ} | 1.64 ± 0.12^{Δ} | 0.35 ± 0.15^{Δ} | 1.35 ± 0.02^{Δ} | 0.36 ± 0.01 | 3.49 ± 0.17^{Δ} | 10.11 ± 0.45^{Δ} |
| 9 | 3.21 ± 0.15^{Δ} | 1.64 ± 0.04^{Δ} | 0.34 ± 0.12^{Δ} | 1.35 ± 0.13^{Δ} | 0.37 ± 0.02 | 3.43 ± 0.15^{Δ} | 10.09 ± 0.64^{Δ} |
| 10 | 3.20 ± 0.24^{Δ} | 1.63 ± 0.08^{Δ} | 0.32 ± 0.03^{Δ} | 1.34 ± 0.11^{Δ} | 0.36 ± 0.10 | 3.33 ± 0.19^{Δ} | 10.09 ± 0.35^{Δ} |
| 11 | 3.20 ± 0.13^{Δ} | 1.63 ± 0.09^{Δ} | 0.32 ± 0.06^{Δ} | 1.33 ± 0.03^{Δ} | 0.36 ± 0.03 | 3.43 ± 0.17^{Δ} | 10.11 ± 0.98^{Δ} |
| 12 | 3.19 ± 0.43^{Δ} | 1.61 ± 0.21^{Δ} | 0.33 ± 0.01^{Δ} | 1.33 ± 0.01^{Δ} | 0.36 ± 0.02 | 3.33 ± 0.23^{Δ} | 10.09 ± 1.11^{Δ} |
| 13 | 3.19 ± 0.22^{Δ} | 1.62 ± 0.24^{Δ} | 0.32 ± 0.09^{Δ} | 1.32 ± 0.01^{Δ} | 0.36 ± 0.01 | 3.13 ± 0.12^{Δ} | 10.09 ± 1.02^{Δ} |

Note:
the sample groups to which the numbers in Table 8 correspond are the same as that in Table 7;
^{Δ}stands for being compared with the high dosage group of Example 1, p < 0.05.

TABLE 9
The comparisons of inflammation-related factors of each group

| | PAI-1 | Adiponectin | CRP | TNF-α | IL-6 | Serum testosterone |
|---|---|---|---|---|---|---|
| No. | (ng/ml) | (ng/ml) | (ng/ml) | (pg/ml) | (pg/ml) | (ng/ml) |
| 1 | 16.83 ± 0.26 | 19.535 ± 10.524 | 1.256 ± 0.262 | 200.117 ± 25.062 | 96.068 ± 9.869 | 9.09 ± 1.99 |
| 2 | 40.67 ± 5.22^{#} | 4.554 ± 0.276^{#} | 5.381 ± 0.253^{#} | 530.200 ± 68.038^{#} | 239.757 ± 17.210^{#} | 1.10 ± 0.12^{#} |
| 3 | 26.89 ± 0.89 | 11.052 ± 1.433 | 3.494 ± 0.894 | 290.481 ± 34.361 | 181.445 ± 24.766 | 3.94 ± 0.51 |
| 4 | 32.96 ± 2.16^{Δ} | 5.924 ± 0.348^{Δ} | 4.057 ± 0.176^{Δ} | 430.869 ± 3.248^{Δ} | 211.334 ± 10.710^{Δ} | 1.17 ± 0.29^{Δ} |
| 5 | 33.22 ± 3.33^{Δ} | 6.021 ± 0.276^{Δ} | 3.991 ± 0.352^{Δ} | 428.667 ± 6.542^{Δ} | 210.346 ± 9.854^{Δ} | 1.12 ± 0.13^{Δ} |
| 6 | 33.16 ± 2.16^{Δ} | 5.924 ± 0.295^{Δ} | 4.133 ± 0.631^{Δ} | 426.869 ± 3.752^{Δ} | 210.232 ± 2.726^{Δ} | 1.27 ± 0.08^{Δ} |
| 7 | 32.87 ± 1.14^{Δ} | 5.936 ± 0.284^{Δ} | 4.124 ± 0.256^{Δ} | 413.869 ± 2.237^{Δ} | 205.235 ± 6.246^{Δ} | 1.17 ± 0.07^{Δ} |
| 8 | 33.00 ± 2.14^{Δ} | 5.956 ± 0.037^{Δ} | 4.112 ± 0.245^{Δ} | 401.869 ± 1.353^{Δ} | 206.235 ± 2.246^{Δ} | 1.29 ± 0.21^{Δ} |
| 9 | 32.12 ± 3.24^{Δ} | 5.927 ± 0.395^{Δ} | 4.027 ± 0.246^{Δ} | 396.869 ± 4.335^{Δ} | 203.346 ± 5.356^{Δ} | 1.34 ± 0.34^{Δ} |
| 10 | 31.89 ± 1.23^{Δ} | 5.968 ± 0.308^{Δ} | 4.036 ± 0.256^{Δ} | 385.869 ± 11.864^{Δ} | 202.245 ± 4.257^{Δ} | 1.37 ± 0.01^{Δ} |
| 11 | 30.99 ± 1.14^{Δ} | 5.947 ± 0.036^{Δ} | 4.002 ± 0.122^{Δ} | 391.869 ± 9.268^{Δ} | 201.146 ± 6.725^{Δ} | 1.40 ± 0.02^{Δ} |
| 12 | 30.98 ± 0.23^{Δ} | 6.036 ± 0.9146^{Δ} | 3.996 ± 0.124^{Δ} | 380.869 ± 6.245^{Δ} | 200.477 ± 4.893^{Δ} | 1.43 ± 0.13^{Δ} |
| 13 | 31.54 ± 2.23^{Δ} | 6.016 ± 0.028^{Δ} | 3.987 ± 0.235^{Δ} | 389.869 ± 3.246^{Δ} | 203.345 ± 5.368^{Δ} | 1.44 ± 0.04^{Δ} |

Note:
the sample groups to which the numbers in Table 9 correspond are the same as that in Table 7;
^{Δ}stands for being compared with the high dosage group of Example 1, p < 0.05.

### Results Analysis

Comprehensive analysis of the above data shows that all indexes in each different combination of single herb are significantly different compared with the high dosage group of Example 1. These results indicate that the formula combination of Example 1 has significant effects on reducing the circumference of waist and abdomen, reducing the visceral fat and anti-inflammation, it is obviously better than other combinations.

The foregoing is only a preferred embodiment(s) of the present disclosure. It should be pointed out that a number of improvements and modifications may also be made by those of ordinary skill in the art without departing from the principles of the present disclosure, and these improvements and modifications are also considered to be within the scope of the present disclosure.

1. A traditional Chinese medicine composition, which is made from:
Poria;
Angelicae Sinensis Radix;
Aurantii Fructus Immaturus; and
Atractylodis Macrocephalae Rhizoma.

2. The traditional Chinese medicine composition according to claim 1, wherein by weight, the dosage of each raw material is:
Poria 10 to 90 parts;
Angelicae Sinensis Radix 5 to 60 parts;
Aurantii Fructus Immaturus 5 to 60 parts; and
Atractylodis Macrocephalae Rhizoma 5 to 60 parts.

3. The traditional Chinese medicine composition according to claim 1, wherein by weight, the dosage of each raw material is:
Poria 20 to 90 parts;
Angelicae Sinensis Radix 10 to 40 parts;
Aurantii Fructus Immaturus 10 to 40 parts; and
Atractylodis Macrocephalae Rhizoma 10 to 40 parts.

4. The traditional Chinese medicine composition according to claim 1, wherein by weight, the dosage of each raw material is:
Poria 30 parts;
Angelicae Sinensis Radix 20 parts;
Aurantii Fructus Immaturus 20 parts; and
Atractylodis Macrocephalae Rhizoma 20 parts.

5. A method for treating abdominal obesity in a subject in need thereof, comprising administering to the subject a therapeutically effective amount of the traditional Chinese medicine composition according to claim 1.

6. The method according to claim 5, wherein the treating abdominal obesity refers to one or more of reducing circumference of waist and abdomen, decreasing Lee's index, or reducing weight of visceral fat, improving the concentrations of inflammation-related factors IL-10, IL-18, PAI-1, adiponectin, TNF-α, IL6 and CRP in serum.

7. A method for preparing the traditional Chinese medicine composition according to claim 1, comprising:
mixing Poria, Angelicae Sinensis Radix, Aurantii Fructus Immaturus and Atractylodis Macrocephalae Rhizoma;
adding water and soaking;
decocting; and
filtering to obtain the traditional Chinese medicine composition.

8. The method according to claim 7, wherein the water is 3 to 10 times by weight as much as the raw materials; soaking time is 0.25 h to 2 h; numbers to decoct is 1 to 5 times; time of each decoction is 1 h to 4 h.

9. A health food, comprising the traditional Chinese medicine composition according to claim 1.
